(19) United States
(12) Patent Application Publication
(10) Pub. No.: US 2014/0120596 A1
(43) Pub. Date: May 1, 2014

(54) PROCESS FOR PRETREATING A LIGNOCELLULOSIC MATERIAL

(71) Applicant: Shell Oil Company, Houston, TX (US)

(72) Inventors: Haisong WANG, Qingdao (CN); Chao Liu, Qingdao (CN); Xindong Mu, Qingdao (CN); Evert Van Der Heide, Amsterdam (NL); Bin Li, Qingdao (CN); Guang Yu, Qingdao (CN)

(73) Assignee: Shell Oil Company, Houston, TX (US)

(21) Appl. No.: 14/066,866

(22) Filed: Oct. 30, 2013

(30) Foreign Application Priority Data
Oct. 31, 2012 (CN) 201210424768.2

(51) Int. Cl. C08B 1/00; C12P 7/14

(52) U.S. Cl. CPC C08B 1/00; C12P 7/14
USPC 435/162; 162/90

(57) ABSTRACT

A process for pretreating a lignocellulosic material is provided. The process comprises mixing the lignocellulosic material and an alkaline aqueous solution, in a solid to liquid weight ratio of equal to or more than 25:75 to equal to or less than 60:40, in a mixer to produce an aqueous slurry and heat-treating the aqueous slurry at a temperature in the range from equal to or more than 50° C. to equal to or less than 200° C. to produce a heat-treated aqueous slurry comprising a pretreated lignocellulosic material. The lignocellulosic material has a lignin content in the range from equal to or more than 10 wt % to equal to or less than 50 wt %, based on the total weight of the lignocellulosic material. The alkaline aqueous solution has a pH of equal to or more than 9.0.

### RELATED APPLICATIONS

This application claims the benefit of Chinese Patent Application No. 201210424768.2, filed on Oct. 31, 2012, the disclosure of which is incorporated herein by reference in its entirety.

### TECHNICAL FIELD

The present disclosure relates to a process for pretreating a lignocellulosic material.

### BACKGROUND

This section is intended to introduce various aspects of the art, which may be associated with exemplary embodiments of the present invention. This discussion is believed to assist in providing a framework to facilitate a better understanding of particular aspects of the present invention. Accordingly, it should be understood that this section should be read in this light, and not necessarily as admissions of any prior art.

With the diminishing supply of crude mineral oil, use of renewable energy sources is becoming increasingly important for the production of fuels and chemicals. These fuels and chemicals from renewable energy sources are often referred to as biofuels, respectively biochemicals. One of the advantages of using renewable energy sources is that the CO2 balance is more favorable as compared with a conventional feedstock of a mineral source.

Biofuels and/or biochemicals derived from non-edible renewable energy sources, such as lignocellulosic material, are preferred as these do not compete with food production. These biofuels and/or biochemicals are also referred to as second generation or advanced biofuels and/or biochemicals. Also for the production of bio-ethanol it would be preferred to produce such from a lignocellulosic material.

A wide variety of methods for converting lignocellulosic material into biofuels and/or biochemicals is available. The digestibility of a lignocellulosic material, however, is hindered by physical, chemical, structural and compositional factors that give the lignocellulosic material the strength that is needed in nature. Many of the conversion methods for lignocellulosic materials therefore have in common that first a pretreatment of the lignocellulosic material is required.

The production of bio-ethanol from a lignocellulosic material may for example comprise as main steps: the pretreatment of the lignocellulosic material to make the cellulose and/or hemicellulose in the lignocellulosic material accessible; hydrolysis of the cellulose and/or hemicellulose to produce sugars; and fermentation of the sugars to bioethanol.

The production of other biofuels and biochemicals, such as for example biodiesel, preferably also comprise a pretreatment step.

Kumar et al. explain in their article titled “Methods for pretreatment of lignocellulosic biomass for efficient hydrolysis and biofuel production,” published in Ind. Eng. Chem. Res. 2009, volume 48, page 3713-3729, that there are several categories of pretreatment methods including: physical (milling and grinding), physicochemical (steam pretreatment/autohydrolysis, hydrothermolysis and wet oxidation), chemical (alkali, dilute acid, oxidizing agents and organic solvents), biological, electrical or a combination of these.

Alvira et al. in their article titled “Pretreatment technologies for an efficient bioethanol production process based on enzymatic hydrolysis: a review,” published in Bioresource Technology 101 (2010), pages 5851-4861 indicate that alkali pretreatments increase cellulose digestibility and that they are more effective for lignin solubilization, exhibiting minor cellulose and hemicellulose solubilization, than acid or hydrothermal processes. As suitable alkaline pretreatments sodium, potassium, and calcium hydroxides are mentioned.

Descriptions in the prior art of alkaline pretreatment methods of a solution of lignocellulosic material with a dry matter content of more than 15 wt % are rare.

Kumar et al. describe a radio-frequency-assisted alkali pretreatment carried out at a 20% solids content and 90° C. with Alkali loadings of 0.1 to 0.25 grams of NaOH per gram of biomass.

Cheng et al. in their article titled “Evaluation of High Solids Alkaline Pretreatment of Rice Straw” describe studies with NaOH done at 55° C. with a water loading of 5 grams water per gram air dried rice straw (i.e. a dry matter content of about 20 wt %) with an alkaline loading of 0.2 and 4 wt % of oven dried rice straw.

Li et al. in their article titled “Cold sodium hydroxide/urea based pretreatment of bamboo for bioethanol production: characterization of the cellulose rich fraction” describe bamboo being ground and sieved to obtain a 40-60 mesh fraction which fraction was extracted with methylbenzene/ethanol to remove fats, waxes and oils and then air dried and subsequently ball-milled. A milled sample was suspended in a 95% ethanol solution for 5 minutes and then subjected to ultrasound irradiation treatment in a ultrasonic cell crusher machine. The sample was filtrated and the residue air dried. The residue was submitted to an alkaline treatment with 7% NaOH/12% urea.

Nlewem et al. in their article titled “Comparison of different pretreatment methods based on residual lignin effect on the enzymatic hydrolysis of switch grass” describe samples of switchgrass being soaked in NaOH solutions of concentrations ranging from 0.5 to 10% w/v at a biomass loading of 0.15 g/ml and a temperature of 85-90° C. for 1 hour.

Chinese Patent Application CN101121175A is directed to the problem that biological or physical or chemical processing of straw takes a long time and the degradation rate is low because the straw lignin and fibrin are hard to be degraded. As a solution it describes a pretreatment method using alkali and ozone. The method includes crushing the straw and mixing it with limewater. In the method, the dry matter of straw is about 3%-15% and the concentration of sodium hydroxide (NaOH) is about 0.3%-1%. The obtained mixture is subsequently treated with ozone.

Chinese Patent Application CN101255479 describes a pretreatment method for saccharifying lignocellulose. In the described pretreatment, ground lignocellulose (20-60 mesh) may be mixed with an alkali solution containing for example sodium hydroxide (NaOH) or potassium hydroxide (KOH) in a concentration of 0.1% to 3%. The liquid solid ratio may lie in the range from 4:1 to 12:1. The described pretreatment process does not appear to need the addition of steam but performs pretreatment at normal temperature condition during about 1 to 4 hours. In the examples solid yields in the range from about 75% to about 93% are obtained. After enzymolysis sugar yields in the range from about 23% to about 53% are obtained. The cellulose conversion rate varied between about 48% and about 51%.

When scaling up to a commercial process, however, the large amounts of water and low dry matter content applied in the processes of the prior art would make the prior art pretreatment processes too expensive to use.

It would therefore be an advancement in the art to provide a pretreatment process that allows one to use a feed having a dry matter content of at least 25 wt %.

Lamsal et al. in their article titled “Extrusion as a thermo-chemical pretreatment for lignocellulosic ethanol,” published in Biomass and Bioenergy vol. 34 (2010) pages 1703-1710, describe the mixing of a solution of sodium hydroxide, urea and thiourea (10 wt % each) with wheat bran to achieve a moisture content of 20-25% w/w (i.e. 75%-80% dry matter) and soybean hull to achieve a moisture content of 30 and 35% w/w (i.e. 65%-70% dry matter). Samples were stored overnight at ambient temperature for equilibration and subsequently extruded at 7 Hz and maximum barrel temperature of 150° C. in a twin-screw extruder. Lamsal et al. explain that the sugar yield from wheat bran, which wheat bran had a lignin content of approximately 7% w/w resulted in a sugar yield from 18 to 20%. Lamsal et al. explain, however, that sugar yields (9-12%) from soybean hull, which soybean hull had a lignin content of 14%, were much lower than wheat bran irrespective of the pre-treatment method. In fact, the preliminary data indicated that extrusion negatively affected sugar release as compared to hammer milling. Lamsal et al. further suggest that extrusion may have led to a greater complexation of the hemicellulose and lignin fractions resulting in even lower sugar yields.

It would be an advancement in the art to address certain challenges related to pretreating a lignocellulosic material.

### SUMMARY

Some embodiments provide for a pretreatment process that allows one to apply a feed with a dry matter content of at least 25 wt % for a lignocellulosic material containing equal to or more than 10 wt % lignin. In one embodiment, there is provided a process for pretreating a lignocellulosic material having a lignin content in the range from equal to or more than 10 wt % to equal to or less than 50 wt %, based on the total weight of the lignocellulosic material, which process comprises: mixing the lignocellulosic material and an alkaline aqueous solution, which alkaline aqueous solution has a pH of equal to or more than 9.0, in a solid to liquid weight ratio of equal to or more than 25:75 to equal to or less than 60:40, in a mixer to produce an aqueous slurry; and heat-treating the aqueous slurry at a temperature in the range from equal to or more than 50° C. to equal to or less than 200° C. to produce a heat-treated aqueous slurry comprising a pretreated lignocellulosic material.

As indicated above, Lamsal et al. suggest that for lignocellulosic materials having a higher lignin content (for example 14 wt %) extrusion may lead to a greater complexation of the hemicellulose and lignin fractions resulting in even lower sugar yields. It has now been found, however, that this does not apply in embodiments provided herein. To the contrary, surprisingly it was found that when applying a solid to liquid weight ratio in the claimed range, improved sugar yields—even as high as 50 wt %—may be obtained.

Other advantages and features of embodiments of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Certain illustrative embodiments of the invention have been illustrated by the following non-limiting following figures:

FIG. 1 shows an exemplary shear mixer that can be used in an embodiment according to aspects the invention.

FIG. 2 shows a schematic flow sheet of one embodiment according to aspects of the invention.

### DETAILED DESCRIPTION

By a lignocellulosic material is herein understood a material containing cellulose, hemicellulose and lignin. Suitably the lignocellulosic material is a solid lignocellulosic material. The lignocellulosic material may be obtained from a wide variety of sources, including for example plants, forestry residues, agricultural residues, herbaceous material, municipal solid wastes, waste and recycled paper, pulp and paper mill residues, sugar processing residues and/or combinations of one or more of the above.

The lignocellulosic material can comprise for example, corn stover, wheat stover, soybean stover, corn cobs, corn fibre, straw (including cereal straws such as wheat straw, barley straw, rye straw and/or oat straw), bagasse, beet pulp, miscanthus, sorghum residue, rice straw, rice hulls, oat hulls, grasses (including switch grass, cord grass, rye grass, reed canary grass or a combination thereof), bamboo, water hyacinth, wood and wood-related materials (including hardwood, hardwood chips, hardwood pulp, softwood, softwood chips, softwood pulp and/or sawdust), waste paper and/or a combination of one or more of these.

The lignocellulosic material is a lignocellulosic material having a lignin content in the range from equal to or more than 10 wt % to equal to or less than 50 wt %, based on the total weight of the lignocellulosic material. The lignocellulosic material preferably has a lignin content of equal to or more than 14 wt %, more preferably equal to or more than 15 wt %, still more preferably equal to or more than 16 wt % and most preferably equal to or more than 17%, based on the total weight of the lignocellulosic material. Further the lignocellulosic material preferably has a lignin content of equal to or less than 40 wt %, more preferably of equal to or less than 35 wt %, and most preferably equal to or less than 30 wt %, based on the total weight of the lignocellulosic material. Preferably the lignocellulosic material contains in the range from equal to or more than 15 wt % lignin to equal to or less than 40 wt % lignin, based on the total weight of the lignocellulosic material. For example, willow wood may contain about 25 wt % lignin, larch wood may contain about 35 wt % lignin, straw may contain about 14 wt % lignin, beech wood may contain about 12-23 wt % lignin and coniferous wood may contain about 25-35 wt % lignin.

Suitably the weight percentages based on the total weight of the lignocellulosic material herein are determined on the total weight of the lignocellulosic material on a dry basis. The lignin may for example include polymers of p-coumaryl alcohol, coniferyl alcohol, sinapyl alcohol, methoxylated coumaryl alcohol, methoxylated coniferyl alcohol, methoxylated sinapyl alcohol and mixtures thereof.

The lignocellulosic material preferably contains equal to or more than 20 wt %, more preferably equal to or more than 30 wt % and most preferably equal to or more than 40 wt % cellulose, based on the total weight of the lignocellulosic material. Preferably the lignocellulosic material contains equal to or less 90 wt %, more preferably equal to or less than 85 wt % cellulose, based on the total weight of the lignocellulosic material. For example the lignocellulosic material may comprise in the range from equal to or more than 20 wt % to equal to or less than 90 wt % cellulose, suitably in the range from equal to or more than 30 wt % to equal to or less than 80 wt % cellulose, based on the total weight of the lignocellulosic material.

In a preferred embodiment, the lignocellulosic material is washed one or more times with water before being supplied to the mixer. The lignocellulosic material may be supplied to the mixer as a wetted lignocellulosic material comprising both lignocellulosic material and water, whereafter the wetted lignocellulosic material is mixed with the alkaline aqueous solution.

Preferably, however, in one embodiment, the lignocellulosic material is supplied to the mixer as a dewatered and/or de-aired lignocellulosic material; and/or the lignocellulosic material is dewatered and/or de-aired during mixing with the alkaline aqueous solution in the mixer (for example in an extruder). This may for example be achieved by drying and/or de-airing of the lignocellulosic material before mixing it with the alkaline aqueous solution. Mixing of the alkaline aqueous solution and a lignocellulosic material, which lignocellulosic material has been dewatered and/or de-aired, may improve penetration of the lignocellulosic material with the alkaline aqueous solution.

If the lignocellulosic material is supplied to the process as a feed comprising both lignocellulosic material and water, the feed preferably has a solid to liquid weight ratio of equal to or more than 25:75 to equal to or less than 60:40 (i.e. the feed contains in the range from equal to or more than 25 wt % to equal to or less than 60 wt % of dry matter).

If the lignocellulosic material is supplied to the process as a dewatered feed, the feed preferably has a solid to liquid weight ratio of equal to or more than 40:60 to equal to or less than 90:10 (i.e. the feed contains in the range from equal to or more than 40 wt % to equal to or less than 90 wt % of dry matter).

In a preferred embodiment, the lignocellulosic material is mixed with an alkaline aqueous solution. By an “alkaline aqueous solution” is herein understood an aqueous solution having a pH of equal to or more than 7.0. In the process according to the invention, the lignocellulosic material is mixed with an alkaline aqueous solution having a pH of equal to or more than 9.0. Preferably the alkaline aqueous solution has a pH of equal to or more than 11.0, more preferably a pH of equal to or more than 12.0. More preferably the alkaline aqueous solution has a pH in the range from equal to or more than 9.0 to equal to or less than 15.0, still more preferably a pH in the range from equal to or more than 11.0 to equal to or less than 14.5, and most preferably a pH in the range from equal to or more than 12.0 to equal to or less than 14.0.

In one embodiment, the alkaline aqueous solution suitably comprises an alkaline pretreatment agent. Preferably such an alkaline pretreatment agent is chosen from the group consisting of sodium hydroxide, potassium hydroxide, sodium carbonate, potassium carbonate sodium sulfide, potassium sulfide and any combination thereof. In a preferred embodiment, the alkaline aqueous solution is an alkaline aqueous solution comprising sodium hydroxide, potassium hydroxide, sodium carbonate, potassium carbonate or mixtures thereof. More preferably, the alkaline aqueous solution is an alkaline aqueous solution containing sodium hydroxide and/or potassium hydroxide. Most preferably, the alkaline aqueous solution is an alkaline aqueous solution containing sodium hydroxide.

If the alkaline aqueous solution is an alkaline aqueous solution containing sodium hydroxide, such alkaline aqueous solution preferably contains in the range from 0.04 gram/liter to 500 gram/liter of sodium hydroxide, more preferably in the range from 4 gram/liter to 450 gram/liter of sodium hydroxide, still more preferably in the range from 10 to 300 gram/liter of sodium hydroxide, and most preferably in the range from 20 to 80 gram/liter of sodium hydroxide.

In a preferred embodiment, the lignocellulosic material and the alkaline aqueous solution are mixed in a solid to liquid weight ratio of equal to or more than 25:75 to equal to or less than 60:40. By a solid to liquid weight ratio of equal to or more than 25:75 to equal to or less than 60:40 is herein preferably understood that an aqueous slurry is formed that appropriately has a dry matter content in the range from equal to or more than 25 wt % to equal to or less than 60 wt %, based on the total weight of the aqueous slurry.

Preferably, the lignocellulosic material and the alkaline aqueous solution are mixed in a solid to liquid weight ratio of equal to or more than 30:70, more preferably equal to or more than 32:68. Further, the lignocellulosic material and the alkaline aqueous solution are preferably mixed in a solid to liquid weight ratio of equal to or less than 55:45. Most preferably, the lignocellulosic material and the alkaline aqueous solution are mixed in a solid to liquid weight ratio of equal to or more than 30:70 to equal to or less than 55:45.

During mixing, a sufficient amount of alkaline aqueous solution and/or additional water and/or steam may be added to bring the solid to liquid weight ratio into the claimed solid to liquid weight ratio. The exact amount of alkaline aqueous solution and/or additional water and/or steam to be added to the mixer may depend on the amount of water that may already be provided in a feed comprising the lignocellulosic material.

In one embodiment, the lignocellulosic material may be supplied to the mixer as a feed comprising both lignocellulosic material and water (herein also referred to as wetted lignocellulosic material), as described in more detail above. When such a wetted lignocellulosic material is used, the wetted lignocellulosic material is preferably mixed in the mixer with a suitable amount of alkaline aqueous solution to provide an aqueous slurry having the claimed solid to liquid weight ratio.

In another embodiment, the lignocellulosic material may be supplied to the process as a dewatered lignocellulosic material as described in more detail above. When such a dewatered lignocellulosic material is used, the dewatered lignocellulosic material is preferably mixed in the mixer with a suitable amount of alkaline aqueous solution and/or additional water and/or steam to provide an aqueous slurry having the claimed a solid to liquid weight ratio.

The lignocellulosic material and alkaline aqueous solution are mixed in a mixer to produce an aqueous slurry. A wide range of mixers may be used including for example extruders, screw mixers, CSTR type mixers, squeezer-type mixers and grinder-type mixers.

In a preferred embodiment, the mixer comprises or consists of one or more a shear mixer(s). By a shear mixer, it is herein preferably understood to refer to a mixer that is suitable to disperse a solid into a liquid phase by applying shear force. In a preferred embodiment, a shear mixer may improve penetration of the internal structure of the lignocellulosic material with the alkaline aqueous solution by applying shear force to the aqueous slurry. By shear force, it is herein preferably understood to refer to a force applied in a direction co-planar to a material cross-section. Application of such shear force may cause one layer of lignocellulosic material to slide over another layer of lignocellulosic material and/or over a fixed surface. The energy applied by the shear mixer is herein referred to as shear energy.

As explained with respect to screw conveyers in R. H. Perry, C. H. Chilton, Chemical Engineers' Handbook, McGraw-Hill Chemical Engineering Series, 5^{th} ed., McGraw-Hill Book Company, New York, 1973, Section 7, p. 6., “required power is made up of two components: that necessary to drive the screw empty and that necessary to move the material. The first component is a function of conveyer length, speed of rotation, and friction in the conveyor bearings. The second is a function of the total weight of material conveyed per unit of time, conveyed length, and depth to which the trough is loaded. The latter power item is in turn a function of the internal friction and friction on metal of the conveyed material.” Preferably shear energy is understood to refer to this internal friction and friction on metal of the conveyed material. Shear energy can be determined by determining the energy needed to operate the mixer after loading the mixer with the lignocellulosic material and subtracting the energy input needed to operate the mixer without loading (i.e. when empty).

In a preferred embodiment the shear energy applied by the shear mixer is in the range of from equal to or more than 1% to equal to or less than 25%, more preferably equal to or more than 2% to equal to or less than 10% of the gross energy contained in the lignocellulosic material. The gross energy (sometimes also referred to as higher heating value HHV or gross calorific value) of the lignocellulosic material is equal to its thermodynamic heat of combustion and can be determined at a standard temperature of 25° C. If so desired, one may for example use ASTM D5865-11a Standard Test Method for Gross Calorific Value of Coal and Coke to determine the gross energy.

In another preferred embodiment, the shear energy applied by the shear mixer lies in the range from equal to or more than 0.05 MegaJoule to equal to or less than 25 MegaJoule per kilogram of lignocellulosic material (as measured at a dry basis). More preferably, the shear energy applied by the shear mixer lies in the range from equal to or more than 0.10 MegaJoule to equal to or less than 2.0 MegaJoule per kilogram of lignocellulosic material (as measured at a dry basis). Most preferably, the shear energy applied by the shear mixer lies in the range from equal to or more than 0.10 to equal to or less than 0.20 MegaJoule per kilogram of lignocellulosic material (as measured at a dry basis).

Preferably the one or more shear mixer(s) comprise or consist of one or more screw mixer(s), one or more extruder(s) or a combination thereof. In one preferred embodiment, the shear mixer comprises or is a screw mixer. The screw mixer preferably comprises a tubular shaped vessel containing a screw. Preferably the tubular shaped vessel is oriented in an essentially horizontal direction. Via the screw, shear energy can be applied to the lignocellulosic material. In a preferred embodiment the shear mixer is a screw reactor. Such a screw reactor comprises a tubular shaped reactor containing a screw. The screw in such a screw reactor may forward the lignocellulosic material from the inlet of the reactor to the outlet of the reactor and in addition mix the lignocellulosic material with a liquid. In one embodiment, the screw can be used to apply shear energy to the lignocellulosic material.

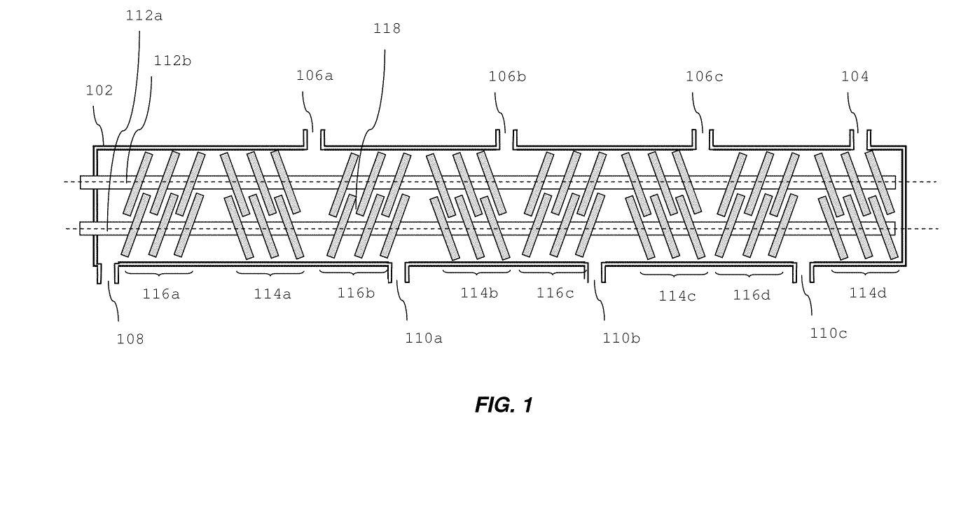

In another preferred embodiment, the shear mixer comprises or is an extruder. Preferably, the extruder is a single screw extruder or a twin screw extruder. Most preferably the shear mixer is a twin screw extruder. A schematic example of a twin screw extruder is provided in FIG. 1, which shows twin screw extruder 102 having solids inlet 104 for lignocellulosic material; several liquid inlets 106a, 106b, and 106c for water and/or an alkaline aqueous solution; slurry outlet 108 for an aqueous slurry and several liquid outlets 110a, 110b, and 110c. Twin screw extruder 102 further comprises two parallel screws 112a and 112b that rotate in the same direction. The helixes of the two screws are forward directed, as referenced by 114a, 114b, 114c, and 114d and reverse directed, as referenced by 116a, 116b, 116c and 116d in an alternate manner. In space 118 between the helixes, shear energy can be applied to the lignocellulosic material and/or the aqueous slurry.

Embodiments provided herein can be carried out without preheating of the lignocellulosic material before mixing it with the alkaline aqueous solution; and/or without applying external heating during the mixing of the lignocellulosic material and the alkaline aqueous solution. Due to the friction between the fibers of the lignocellulosic material and the friction between the lignocellulosic material and the mixer, a substantial amount of friction heat may be generated. In the process of the invention, friction heat that may be in-situ generated during the mixing of the lignocellulosic material and the alkaline aqueous solution may be used to increase the temperature. In a particular embodiment, the use of a shear mixer increases the amount of friction heat that may be generated. If the mixer is a shear mixer the friction heat may be generated by the shear energy as described herein before, and the friction heat may also be referred to as shear energy.

If so desired, external heating may be applied in addition to the friction heat. In one embodiment, the amount of external heating may be limited to providing a further temperature increase (ΔT) in the range from equal to or more than 5° C. to equal to or less than 100° C., more preferably in the range equal to or more than 5° C. to equal to or less than 30° C.

In one embodiment, the lignocellulosic material and optionally water may be supplied to a shear mixer; after dewatering and de-airing the lignocellulosic material within such shear mixer, the lignocellulosic material may be mixed with the alkaline aqueous solution in the same or another shear mixer. Preferably, the shear mixer for this embodiment is a twin screw extruder as illustrated in FIG. 1, wherein an optionally wetted lignocelullosic material is supplied at solids inlet 104; a part of water is added to the lignocellulosic material at a first liquid inlet 106c along the extruder; at least part of the water is removed via one or more liquid outlets 110c and 110b of extruder 102; followed by one or more parts of an alkaline aqueous solution being added to the lignocellulosic material at one or more subsequent liquid inlets (for example at 106a and/or 106b) along extruder 102. In this embodiment, outlet 110a is preferably closed before liquid inlet 106b is opened for addition of any alkaline aqueous solution, to avoid the unnecessary loss of alkaline aqueous solution. In one embodiment, residual alkaline aqueous solution and/or water and/or any dissolved lignin may be removed via one or more of the liquid outlets 110a and/or 110b. The aqueous slurry comprising the pretreated lignocellulosic material can be obtained from outlet 108.

In one embodiment, the alkaline aqueous solution is fed to the mixer in a staged manner. For example, the alkaline aqueous solution may be added to the mixer via two or more subsequent inlets. More preferably, the alkaline aqueous solution is fed to the mixer via two or more subsequent inlets; and water and/or residual alkaline aqueous solution and/or any dissolved lignin is removed from the mixer via two or more subsequent outlets. Feeding the alkaline aqueous solution to the mixer in a staged manner may result in a better wetting of the lignocellulosic material. In addition, removal of water and/or residual alkaline aqueous solution and/or any dissolved lignin via two or more outlets may result in an improved removal of for example dirt and soil.

Mixing of the lignocellulosic material and the alkaline aqueous solution in the mixer may be carried out at a wide range of temperatures and pressures. In a preferred embodiment, the mixing is carried out at a temperature in the range from equal to or more than 50° C. to equal to or less than 150° C., more preferably equal to or more than 50° C. to equal to or less than 120° C., still more preferably at a temperature in the range from equal to or more than 65° C. to equal to or less than 100° C., and most preferably at a temperature in the range from equal to or more than 90° C. to equal to or less than 96° C. Preferably, the mixing is carried out at a pressure in the range from equal to or more than 0.1 MegaPascal (about 1 bar) to equal to or less than 2.0 MegaPascal (about 20 bar), more preferably a pressure of about 0.1 MegaPascal.

Further, the residence time of the lignocellulosic material in the mixer may vary widely. Preferably this residence time of the lignocellulosic material in the mixer is equal to or more than 10 seconds, more preferably equal to or more than 20 seconds, still more preferably equal to or more than 1 minutes, and most preferably equal to or more than 2 minutes. Preferably, the residence time of the lignocellulosic material in the mixer is further equal to or less than 10 hours, more preferably equal to or less than 5 hours, still more preferably equal to or less than 1 hour, and most preferably equal to or less than 30 minutes. More preferably the residence time of the lignocellulosic material in the mixer ranges from equal to or more than 10 seconds to equal to or less than 2 hours, even more preferably from equal to or more than 20 seconds to equal to or less than 1 hour. Most preferably the residence time ranges from equal to or more than 1 minute to equal to or less than 20 minutes.

In the mixer, an aqueous slurry is produced. The aqueous slurry preferably comprises pretreated lignocellulosic material, water, and optionally some dissolved alkaline pretreatment agent. Preferably, the aqueous slurry has a dry matter content in the range from equal to or more than 25 wt % to equal to or less than 60 wt %, based on the total weight of the aqueous slurry, more preferably a dry matter content in the range from equal to or more than 30 wt % to equal to or less than 55 wt %, based on the total weight of the aqueous slurry. Further the aqueous slurry preferably has a pH of equal to or more than 9.0, more preferably of equal to or more than 10.0 and most preferably of equal to or more than 11.0. Most preferably the aqueous slurry has a pH in the range from equal to or more than 9.0 or more than 11.0 to equal to or less than 14.0.

In one embodiment, the aqueous slurry is heat-treated at a temperature in the range from equal to or more than 50° C. to equal to or less than 200° C. The pressure during this heat-treatment is preferably similar to the pressure in the mixer and is preferably a pressure in the range from equal to or more than 0.1 MegaPascal (1 bar) to equal to or less than 2.0 MegaPascal (20 bar), more preferably a pressure of about 0.1 MegaPascal. The mixing of the lignocellulosic material and the alkaline aqueous solution and the heat-treating can be carried out in sequence or simultaneously.

In one embodiment, the mixing and heat-treating are carried out simultaneously in one step and provides a process for pretreating a lignocellulosic material having a lignin content in the range from equal to or more than 10 wt % to equal to or less than 50 wt %, based on the total weight of the lignocellulosic material, which process comprises: mixing the lignocellulosic material and an alkaline aqueous solution, which alkaline aqueous solution has a pH of equal to or more than 9.0, at a temperature in the range from equal to or more than 50° C. to equal to or less than 200° C., more preferably to equal to or less than 150° C., in a solid to liquid weight ratio of equal to or more than 25:75 to equal to or less than 60:40, in a mixer to produce a heat-treated aqueous slurry comprising a pretreated lignocellulosic material. This process is hereafter also referred to as one-step process. Preferences for such a one-step process, for example preferences for the mixer and the aqueous slurry, are as described herein above in general. In one embodiment, preferably the mixer in such a one-step process is a shear mixer as described herein above.

In such a one-step process the residence time of the lignocellulosic material in the mixer more preferably ranges from equal to or more than 20 seconds to equal to or less than 10 hours, even more preferably from equal to or more than 1 minute to equal to or less than 5 hours; and most preferably from equal to or more than 20 minutes to equal to or less than 2 hours.

In another embodiment, the mixing and heat-treating are carried out in a two-step process and provides a process for pretreating a lignocellulosic material having a lignin content in the range from equal to or more than 10 wt % to equal to or less than 50 wt %, based on the total weight of the lignocellulosic material, which process comprises: mixing the lignocellulosic material and an alkaline aqueous solution, which alkaline aqueous solution has a pH of equal to or more than 9.0, in a solid to liquid weight ratio of equal to or more than 25:75 to equal to or less than 60:40, in a mixer to produce an aqueous slurry; transporting the aqueous slurry from the mixer into a reaction vessel; and heat-treating the aqueous slurry in the reaction vessel at a temperature in the range from equal to or more than 50° C. to equal to or less than 200° C. to produce a heat-treated aqueous slurry comprising a pretreated lignocellulosic material. This process is hereafter also referred to as two-step process. Preferences for such a two-step process, for example preferences for the mixer and the aqueous slurry, are as described herein above in general.

Preferably the mixer in such a two-step process is a shear mixer as described herein above. In such a two-step process, the temperature applied during the mixing step may differ from the temperature applied during the heat-treating step. In the two-step process, the lignocellulosic material and the alkaline aqueous solution are preferably mixed in the mixer at a temperature equal to or below 150° C., more preferably at a temperature equal to or below 120° C. and even more preferably at a temperature equal to or below 100° C. Still more preferably, the lignocellulosic material and the alkaline aqueous solution are mixed in the mixer at a temperature in the range from equal to or more than 20° C. to equal to or less than 120° C., even more preferably in the range from equal to or more than 50° C. to equal to or less than 120° C. and still more preferably in the range from equal to or more than 50° C. to equal to or less than 100° C. Most preferably, the two-step process comprises mixing of the lignocellulosic material and the alkaline aqueous solution at a temperature in the range from equal to or more than 80° C. to equal to or less than 96° C. The pressure during the mixing step in the two-step process is preferably about 0.1 MegaPascal.

The temperature during the heat-treating step in the two-step process is preferably equal to or higher than the temperature during the mixing step. In the two-step process the aqueous slurry is preferably heat-treated in the reaction vessel at a temperature in the range from equal to or more than 65° C. to equal to or less than 200° C., more preferably in the range from equal to or more than 100° C. to equal to or less than 150° C. The pressure during the heat-treating step in the two-step process is preferably a pressure in the range from equal to or more than 0.0 to equal to or less than 1.6 MegaPascal.

Such a two-step process may further have the advantages that the residence time in the mixer may be reduced allowing for a higher throughput during commercial operation. Preferably the residence time in the mixer is equal to or lower than the residence time in the reaction vessel.

In the above two-step process the residence time of the lignocellulosic material in the mixer preferably ranges from equal to or more than 10 seconds to equal to or less than 2 hours, more preferably from equal to or more than 20 seconds to equal to or less than 1 hour, and most preferably from equal to or more than 1 minutes to equal to or less than 20 minutes. The residence time of the aqueous slurry in the reaction vessel may vary widely. Preferably the residence time of the aqueous slurry in the reaction vessel ranges from equal to or more than 1 minute to equal to or less than 10 hours, more preferably from equal to or more than 5 minutes to equal to or less than 5 hours.

The total residence time of the aqueous slurry in the mixer and any reaction vessel (i.e. the combined residence time) preferably lies in the range from equal to or more than 20 seconds to equal to or less than 10 hours, more preferably in the range from equal to or more than 1 minute to equal to or less than 5 hours, and most preferably from equal to or more than 20 minutes to equal to or less than 2 hours.

The aqueous slurry may be transported from the mixer into the reaction vessel by any transporting means known to the skilled person to be suitable for this purpose. These transporting means may for example include pipes, pumps, conveyer belts or other types of conveyers. In a preferred embodiment, the aqueous slurry is transported from the mixer into the reaction vessel with the help of one or more screw pumps. During the transport from the mixer into the reaction vessel, the aqueous slurry may or may not be heated to keep the aqueous slurry at a certain temperature. In addition the transporting means may be heat-insulated to prevent leakage of heat from the process.

The reaction vessel may be any vessel known to the person skilled in the art to be suitable for carrying out a reaction. It may for example be an extruder, plug flow reactor, screw reactor, continuously stirred tank reactor (CSTR), Pandia digester, moving belt reactor or L&G type reactor. Also combinations thereof may be used. Preferably the reaction vessel is a vessel that can be heated and/or insulated and/or pressurized. In one preferred embodiment the reaction vessel may be an adiabatic reaction vessel, where the heat generated during the mixing is used as the main heating source. In another preferred embodiment the reaction vessel may be a heat insulated reaction vessel to prevent heat from leaking away.

In the two-step process, friction heat that may be in-situ generated during the mixing of the lignocellulosic material and the alkaline aqueous solution may be used to increase the temperature of the aqueous slurry. In one embodiment, it is therefore not needed to add any external heat to the reaction vessel. In this embodiment, friction heat generated during the mixing in the mixer may be used as the sole heat source for the heat-treating in the reaction vessel.

In another embodiment, heat is supplied to the reaction vessel by an external heat source. In this case, a first part of the heat needed for the heat-treating may be supplied by the friction heat generated during the mixing, whilst a second part of the heat needed for the heat-treating is supplied to the reaction vessel by an external heat source. A preferred external heat source is steam. The addition of extra heat may accelerate the pretreatment.

The reaction vessel may contain mechanical displacement means. In a preferred embodiment however, the reaction vessel contains no or only limited displacement means. Examples of mechanical displacement means include stirrers or screws.

In the above two-step process, one, two or more reaction vessels may be used. If two or more reaction vessels are used, such two or more reaction vessels may be any combination of the reaction vessels as described above. In a preferred embodiment, the aqueous slurry is transported into and heat-treated in two or more reaction vessels. Such two or more reaction vessels may be applied in parallel or in sequence. The use of two or more reaction vessels allows one to operate the mixer in a continuous manner. For example, when the residence time in the mixer is lower than the residence time in the reaction vessel, the use of two or more reaction vessels in parallel may allow one to fill up a subsequent reaction vessel, whilst a previous reaction vessel is still being used for the heat-treatment or whilst a previous reaction vessel is being emptied. The use of two or more reaction vessels in sequence may allow one to spread the residence time for the heat-treatment over multiple reaction vessels, also allowing for continuous operation.

During heat-treating of the aqueous slurry, a heat-treated aqueous slurry is produced. The heat-treated aqueous slurry preferably comprises pretreated lignocellulosic material, water, optionally dissolved lignin and optionally some dissolved alkaline pretreatment agent. Preferably, the heat-treated aqueous slurry has a dry matter content in the range from equal to or more than 25 wt % to equal to or less than 60 wt %, based on the total weight of the aqueous slurry, more preferably a dry matter content in the range from equal to or more than 30 wt % to equal to or less than 55 wt %, based on the total weight of the heat-treated aqueous slurry.

The heat-treated aqueous slurry may be further processed in one or more subsequent steps before the pretreated lignocellulosic material contained therein is converted into an end-product. For example, in a subsequent step, part of the water and optionally any dissolved lignin may be removed from the heat-treated aqueous slurry. Water may for example be at least partly removed with the help of a filter, a cyclone, drum vacuum washer, horizontal belt washer.

The pretreated lignocellulosic material produced in the process according to the invention may be subjected to other steps, such as for example a neutralization step, a steam blasting step or a hydrolysis step.

In a preferred embodiment the pretreated lignocellulosic material produced in the process according to the invention is converted into a biofuel and/or biochemical. For example the pretreated lignocellulosic material can be converted to one or more hydrocarbons, for example hydrocarbons comprising in the range from 6 to 20 carbon atoms. Such hydrocarbons can for example be useful as a component in a gasoline and/or diesel fuel or in a lubricant. The pretreated lignocellulosic material may also be converted to one or more alkanol(s), for example ethanol and/or butanol. Most preferably the pretreated lignocellulosic material produced in the process according to the invention is converted into ethanol.

Depending on the pH of the pretreated lignocellulosic material and the envisaged subsequent processing steps, in one embodiment, the pretreated lignocellulosic material may be neutralized to a pH in the range from equal to or more than 4.0 to equal to or less than 7.0. Such a neutralization may for example be carried out by means of washings with water and/or washings with a limited amount of an acidic aqueous solution. The neutralized pretreated lignocellulosic material may conveniently be converted to one or more alkanol(s), such as for example ethanol and/or butanol with the help of enzymatic hydrolysis.

In one embodiment, there is also provided a process for the production an alkanol, preferably ethanol, comprising a) pretreating a lignocellulosic material having a lignin content in the range from equal to or more than 10 wt % to equal to or less than 50 wt %, based on the total weight of the lignocellulosic material, which pretreating comprises mixing the lignocellulosic material and an alkaline aqueous solution, which alkaline aqueous solution has a pH of equal to or more than 9.0, in a solid to liquid weight ratio of equal to or more than 25:75 to equal to or less than 60:40, in a mixer to produce an aqueous slurry; and heat-treating the aqueous slurry at a temperature in the range from equal to or more than 50° C. to equal to or less than 200° C. to produce a heat-treated aqueous slurry containing pretreated lignocellulosic material. The process further comprises b) converting the pretreated lignocellulosic material into an alkanol, preferably ethanol. Preferences for step a) are as described herein above.

In addition to the pretreated lignocellulosic material, the heat-treated aqueous slurry obtained from step a) may contain water. In a preferred embodiment, at least part of the water and optionally any dissolved lignin is removed from the heat-treated aqueous slurry containing the pretreated lignocellulosic material before subjecting the pretreated lignocellulosic material to step b).

In a further preferred embodiment the pretreated lignocellulosic material obtained from step a) is washed and/or neutralized before subjecting it to step b). As explained above, in one embodiment, the pH of the pretreated lignocellulosic material is neutralized to a pH in the range from equal to or more than 4.0 to equal to or less than 7.0, before subjecting it to step b).

In a preferred embodiment, step b) comprises i) hydrolyzing at least part of the, optionally neutralized, pretreated lignocellulosic material produced in step a) to produce a hydrolysis product; and ii) fermenting at least part of the hydrolysis product produced in step i) to produce a fermentation broth comprising the one or more alkanol(s).

In a preferred embodiment, such steps i) and ii) are followed by: an optional step iii) comprising retrieving the one or more alkanols from the fermentation broth produced in step ii).

The hydrolysis in step i) may be carried out in any manner known to the skilled person in the art to be suitable for the hydrolysis of a lignocellulosic material. Preferably the, optionally neutralized, pretreated lignocellulosic is hydrolyzed in step i) by enzymatic hydrolysis. In an especially preferred embodiment step i) comprises hydrolyzing the, optionally neutralized, pretreated lignocellulosic material with the help of one or more cellulase enzymes, one or more glucosidase enzymes, one or more xylanase enzymes or a combination thereof. By hydrolysis of the optionally neutralized pretreated lignocellulosic material a hydrolysis product is produced. The hydrolysis product may contain one or more sugars. The sugars may comprise for example monosaccharides and disaccharides. For example the hydrolysis product may contain glucose, xylose, galactose, mannose, arabinose, fructose, rhamnose and/or mixtures thereof.

In step ii) at least part of the hydrolysis product produced in step i) can be fermented to produce a fermentation broth. The fermentation in step ii) may for example be carried out with the help of a microorganism. The microorganism may be any kind of microorganism known to be capable of fermenting part or whole of the hydrolysis product. For example, it may be a microorganism capable of fermenting part or whole of the hydrolysis product to a fermentation broth containing ethanol and/or butanol. Preferably the microorganism is a yeast or bacterium. More preferably the microorganism is chosen from the group consisting of Saccharomyces spp., Saccharomyces cerevisiae, Escherichia, Zymomonas, Candida, Pichia, Streptomyces, Bacillus, Lactobacillus, Clostridium and mixtures thereof.

In one embodiment, the hydrolyzing of step i) and the fermentation of step ii) are carried out simultaneously in the same reactor. It is, however, most preferred to carry out the hydrolyzing of step i) and the fermentation of step ii) separately to allow for optimal temperatures for each step. The fermentation broth generated in step ii) may contain one or more alkanols. Preferably the fermentation broth contains ethanol and/or butanol. Most preferably the fermentation broth is a fermentation broth containing ethanol.

In optional step iii), the one or more alkanols are retrieved from the fermentation broth produced in step ii). Preferably step iii) comprises distillation of the fermentation broth to produce one or more distillation fraction(s) comprising the one or more alkanol(s), for example a distillation fraction comprising ethanol and/or a distillation fraction comprising butanol and/or a distillation fraction comprising ethanol and butanol.

The one or more alkanol(s), for example the butanol and/or ethanol, may be blended with one or more other components to produce a biofuel or a biochemical. Examples of one or more other components with which the one or more alkanol(s) may be blended include anti-oxidants, corrosion inhibitors, ashless detergents, dehazers, dyes, lubricity improvers and/or mineral fuel components and/or other fuel components, such as for example so-called Fischer-Tropsch derived fuel components or other renewable fuel components.

In one embodiment, there is also provided a process for the production of a fuel comprising steps a) and b) as described herein above and further comprising an additional step of blending the one or more alkanols produced in step b) with one or more other fuel components to produce a fuel.

Unless indicated otherwise, any weight percentages described herein are determined on a dry basis, i.e. after removal of any residual water.

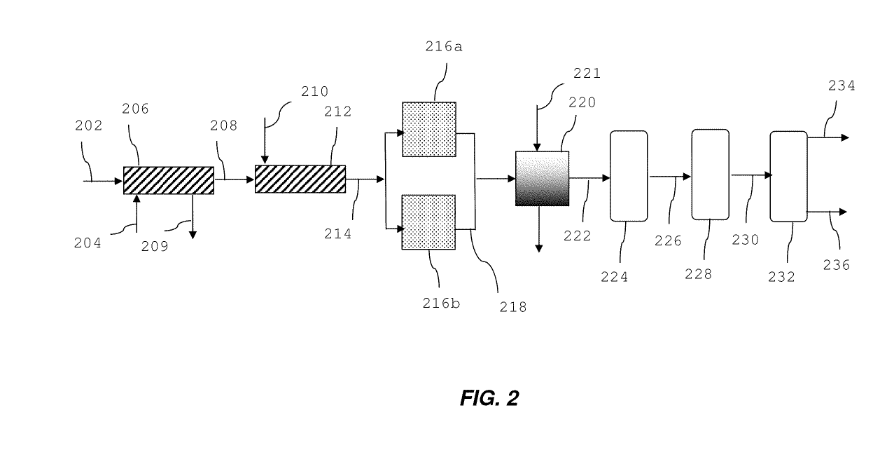

FIG. 2 shows a schematic flow sheet of one embodiment according to aspects of the invention. In FIG. 2, lignocellulosic material 202 is wetted and washed with water 204 in first screw mixer 206 to produce wetted lignocellulosic material composition 208 and waste water 209. Wetted lignocellulosic material composition 208 is mixed with alkaline aqueous solution 210 in second screw mixer 212 at a temperature of 90° C. for 10 minutes. Aqueous slurry 214 having a solid to liquid weight ratio of 35:65 is obtained from second screw mixer 212. Aqueous slurry 214 is forwarded to at least one of two heat-insulated reaction vessels 216a and 216b. In heat-insulated reaction vessels 216a and/or 216b, aqueous slurry 214 is heat-treated for 3 hours to produce heat-treated aqueous slurry 218 containing pretreated lignocellulosic material and water. Heat-treated aqueous slurry 218 is subsequently forwarded to purification and neutralization unit 220 where the pretreated lignocellulosic material is retrieved from heat-treated aqueous slurry 218 and subsequently washed with water 221 in neutralization unit 220 to produce neutralized pretreated lignocellulosic material 222. Neutralized pretreated lignocellulosic material 222 is thereafter forwarded to hydrolysis unit 224 where neutralized pretreated lignocellulosic material 222 is hydrolyzed with the help of cellulase enzymes to produce hydrolysis product 226 containing sugars. The sugars in hydrolysis product 226 are fermented in fermentation unit 228 with the help of a fermentation microorganism to produce fermentation broth 230 containing an alcohol, such as ethanol. Fermentation broth 230 is subsequently distilled in distiller 232 to produce ethanol 234 and other products 236. Some embodiments provided herein are further illustrated by the following non-limiting examples.

### EXAMPLES

### Example 1

Air dried corn stover having a lignin of about 21 wt % on a dry basis was cut into 3-5 cm pieces and loaded into a twin screw extruder (TEP80 commercially obtainable from Hebei Tianzheng Screening pulp-making Device Co. Ltd) via its feed inlet. Water was added via the feed inlet for washing and the water content of the squeezed material was adjusted via the liquid outlet to produce an aqueous slurry having a dry matter concentration of about 50 wt % (corresponding to a solid to liquid weight ratio of 50:50). An aqueous sodium hydroxide solution having a concentration of about 60 grams sodium hydroxide per litre was added via a liquid inlet. This aqueous sodium hydroxide solution had a pH of about 14.2.

The amount of aqueous sodium hydroxide solution was controlled so that the dry matter content of the slurry was reduced to about 33 wt %, corresponding to a solid to liquid weight ratio of 33:67. (i.e. the weight of the added sodium hydroxide solution was equal to 1 time the weight of dry matter already present in the aqueous slurry). The main shaft rotation speed of the twin screw extruder was set as 375 rpm. An aqueous slurry having a dry matter content of about 33 wt % was collected from the discharge outlet of the twin screw extruder and was placed in a heat insulated reaction vessel during 3 hours. The temperature in the twin screw extruder and heat-insulated reaction vessel during pretreatment was about 94° C.

The reaction conditions are summarized in Table 1 below.

After standing, the material was washed to be neutral, then its chemical components were analyzed according to the methods as described in NREL/TP-510-42618 (http://www.nrel.gov/biomass/pdfs/42618.pdf), and a high performance liquid chromatography (HPLC, Model 1200, Agilent Technologies, USA) was used according to the instructions from the manufacturer to detect its carbohydrate content.

The pretreatment recovery rate (i.e. weight of the residue obtained) on the basis of the initial material, and the weight percentages of respectively glucan, xylan and lignin in the residue obtained are listed in Table 2 below. This indicated that after the pretreatment according to embodiments provided by the disclosure, the lignin in the corn stalk was effectively removed.

Under pH 4.8 and 50° C., cellulase (Celluclast 1.5 L) of 20 FPU/g substrate and β-glucosidase (Novozyme 188) of 5 IU/g substrate were used to hydrolyze the pretreated material in 2% substrate concentration for 48 h. A high performance liquid chromatography (HPLC, Model 1200, Agilent Technologies, USA) was used according to the instructions of the manufacturer to analyze the hydrolysis product, and the hydrolysis efficiency of glucose, the hydrolysis efficiency of xylose and the total sugar yield were calculated.

The hydrolysis efficiency of glucose; the hydrolysis efficiency of xylose and the total sugar yield are listed in Table 2. The results indicate that the corn stover could be easily enzymatically degraded after the pretreatment according to embodiments provided by the present disclosure, and both the hydrolysis efficiencies and the total sugar yield were high.

### Example 2

Example 2 was carried out in a similar manner as example 1, except as indicated herein below. The air dried corn stover having a lignin of about 21 wt % on a dry basis was shredded by a shredder into 1-3 cm pieces. Water was added via the feed inlet for washing and the water content of the squeezed material was adjusted via the liquid outlet to produce an aqueous slurry having a dry matter concentration of about 45 wt % (corresponding to a solid to liquid weight ratio of 45:55). An aqueous sodium hydroxide solution having a concentration of about 100 grams sodium hydroxide per litre was added via a liquid inlet. This aqueous sodium hydroxide solution had a pH of about 14.4. The amount of aqueous sodium hydroxide solution was controlled so that the dry matter content of the aqueous slurry was reduced to about 35 wt %, corresponding to a solid to liquid weight ratio of 35:65. (i.e. the weight of the added sodium hydroxide solution was equal to 0.6 time the weight of dry matter already present in the aqueous slurry). An aqueous slurry having a dry matter content of about 35% w/w was collected from the discharge outlet of the twin screw extruder and was placed in a heat insulated reaction vessel during 1 hours. The temperature in the twin screw extruder and heat-insulated reaction vessel during pretreatment was about 98° C.

The reaction conditions are summarized in Table 1 below. The pretreatment recovery rate (i.e. weight of the residue obtained) on the basis of the initial material, and the weight percentages of respectively glucan, xylan and lignin in the residue obtained are listed in Table 2 below. Also the hydrolysis efficiency of glucose; the hydrolysis efficiency of xylose and the total sugar yield are listed in Table 2.

### Example 3

Poplar wood pieces with a lignin content of about 23 wt % on a dry basis and a size of about 4 cm*4 cm were washed with water, then loaded into a twin screw extruder (TEP80 from Hebei Tianzheng Screening Pulp-making Device Co., Ltd.) via its feed inlet. The first 2 squeezing zones of the twin screw extruder were used to remove a part of water and gas from the wood pieces so that the material had a dry matter content of about 60 wt % (corresponding to a solid to liquid weight ratio of 60:40).

An aqueous sodium hydroxide solution with a concentration of about 430 grams sodium hydroxide per litre was added via a liquid inlet. This aqueous sodium hydroxide solution had a pH of about 15.0. The amount of sodium hydroxide solution was controlled so that the dry matter content was further decreased to about 40% (i.e., the weight of the used aqueous sodium hydroxide solution was equivalent to 0.83 times the weight of absolute dry material).

The main shaft rotation speed of the twin screw extruder was set as 350 rpm. The pretreated material with a dry matter concentration of about 40 wt % was collected from the discharge outlet. The temperature of the material at the discharge outlet of the extruder was measured as 98° C. The material was placed in a thermal insulation tank for 1 hour. The reaction conditions are summarized in Table 1 below.

For the remainder example 3 was carried out as example 1. The pretreatment recovery rate (i.e. weight of the residue obtained) on the basis of the initial material, and the weight percentages of respectively glucan, xylan and lignin in the residue obtained are listed in Table 2 below. Also the hydrolysis efficiency of glucose; the hydrolysis efficiency of xylose and the total sugar yield are listed in Table 2.

TABLE 1
Reaction conditions of examples 1 to 3.

| Example | 1 | 2 | 3 |
|---|---|---|---|
| Lignin content of the LM (wt %) | 21 | 21 | 23 |
| NaOH (grams/liter) in AAS and pH of | 6 (13.2) | 10 (13.4) | 43 (14.0) |
| the solution | | | |
| Dry matter in aqueous slurry after | 33 | 35 | 40 |
| addition of AAS (wt %) | | | |
| Temperature in twin screw extruder | 94 | 98 | 98 |
| (° C.) | | | |
| Temperature in heat-insulated reaction | 94 | 98 | 98 |
| vessel | | | |
| Total energy consumption* | 0.21 | 0.20 | 0.25 |
| (MegaJoule/kilogram lignocellulosic | | | |
| material) | | | |
| Shear energy** | 0.16 | 0.15 | 0.20 |
| (MegaJoule/kilogram lignocellulosic | | | |
| material) | | | |
| Total Residence time at reaction | 3 | 1 | 1 |
| conditions, including both the residence | | | |
| time in the extruder and in the reactor | | | |
| vessel (hours) | | | |

LM = lignocellulosic material
AAS = alkaline aqueous solution
*Energy consumption is the energy required to operate the twin screw extruder, this includes the shear energy.
**calculated by subtracting the energy consumed when the extruder was running empty (i.e. 0.05 MegaJoule/Kilogram lignocellulosic material) from the total energy consumption.

TABLE 1
Product characteristics of examples 1 to 3

| Example | 1 | 2 | 3 |
|---|---|---|---|
| Pretreatment recovery rate (wt % based | 63.6 | 61.7 | 78.6 |
| on the weight of the initial material) | | | |
| glucan (wt % on residue) | 55.2 | 61.5 | 51.8 |
| glucan (wt % on initial material) | 35.1 | 37.9 | 40.7 |
| xylan (wt % on residue) | 22.5 | 21.0 | 19.2 |
| xylan (wt % on initial material) | 14.3 | 13.0 | 15.1 |
| lignin (wt % on residue) | 8.6 | 8.6 | 20.3 |
| lignin (wt % on initial material) | 5.5 | 5.3 | 16.0 |
| hydrolysis efficiency of glucose (wt % on | 81.3 | 89.0 | 85.3 |
| glucan in the residue) | | | |
| Yield of glucose (wt % on initial material) | 28.5 | 33.7 | 34.7 |
| hydrolysis efficiency of xylose (wt % on | 88.4 | 84.6 | 78.1 |
| xylan in the residue) | | | |
| Yield of xylose (wt % on initial material) | 12.6 | 11.0 | 11.8 |
| Total sugar yield (wt % on initial | 46 | 50 | 52 |
| material) | | | |

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A process for pretreating a lignocellulosic material comprising:
mixing a lignocellulosic material and an alkaline aqueous solution in a solid to liquid weight ratio of equal to or more than 25:75 to equal to or less than 60:40 in a mixer to produce an aqueous slurry, wherein the lignocellulosic material has a lignin content in the range from equal to or more than 10 wt % to equal to or less than 50 wt %, based on the total weight of the lignocellulosic material, and the alkaline aqueous solution has a pH of equal to or more than 9.0; and
heat-treating the aqueous slurry at a temperature in the range from equal to or more than 50° C. to equal to or less than 200° C. to produce a heat-treated aqueous slurry comprising a pretreated lignocellulosic material.

2. The process of claim 1, wherein the lignocellulosic material contains in the range from equal to or more than 15 wt % lignin to equal to or less than 40 wt % lignin, based on the total weight of the lignocellulosic material.

3. The process of claim 1, wherein the alkaline aqueous solution is an alkaline aqueous solution comprising sodium hydroxide, potassium hydroxide, sodium carbonate, potassium carbonate or any combination thereof.

4. The process of claim 1, wherein the alkaline aqueous solution comprises sodium hydroxide in the range from 0.04 gram/liter to 500 gram/liter.

5. The process of claim 1, wherein the lignocellulosic material and the alkaline aqueous solution are mixed in a solid to liquid weight ratio of equal to or more than 30:70 to equal to or less than 55:45.

6. The process of claim 1, wherein the mixing comprises applying a shear force.

7. The process of claim 6, wherein an amount of shear energy applied is in a range of equal to or more than 1% to equal to or less than 25% of the gross energy contained in the lignocellulosic material.

8. The process of claim 6, wherein an amount of shear energy applied is in a range from equal to or more than 100 Joule to equal to or less than 20,000 Joule per gram of lignocellulosic material.

9. The process of claim 1, wherein the mixer is a twin screw extruder.

10. The process of claim 1, wherein residence time of the lignocellulosic material in the mixer ranges from equal to or more than 10 seconds to equal to or less than 10 hours.

11. The process of claim 1, wherein the mixing and the heat-treating are carried out in sequence.

12. The process of claim 1, wherein it mixing and the heat-treating are carried out simultaneously.

13. The process of claim 1, which process comprises mixing the lignocellulosic material and an alkaline aqueous solution, which alkaline aqueous solution has a pH of equal to or more than 9.0, at a temperature in the range from equal to or more than 50° C. to equal to or less than 200° C., in a solid to liquid weight ratio of equal to or more than 25:75 to equal to or less than 60:40, in a mixer to produce a heat-treated aqueous slurry comprising a pretreated lignocellulosic material

14. The process of claim 1 further comprises:
transporting the aqueous slurry from the mixer into a reaction vessel; and
heat-treating the aqueous slurry in the reaction vessel at a temperature in the range from equal to or more than 50° C. to equal to or less than 200° C. to produce a heat-treated aqueous slurry comprising a pretreated lignocellulosic material.

15. The process of claim 13, wherein the residence time in the mixer is equal to or lower than the residence time in the reaction vessel.

16. A process for the production of an alkanol comprising
pretreating a lignocellulosic material having a lignin content in the range from equal to or more than 10 wt % to equal to or less than 50 wt %, based on the total weight of the lignocellulosic material by mixing the lignocellulosic material and an alkaline aqueous solution in a mixer to produce an aqueous slurry; wherein the alkaline aqueous solution has a pH of equal to or more than 9.0, in a solid to liquid weight ratio of equal to or more than 25:75 to equal to or less than 60:40;
heat-treating the aqueous slurry at a temperature in the range from equal to or more than 50° C. to equal to or less than 200° C. to produce a heat-treated aqueous slurry containing pretreated lignocellulosic material; and
converting the pretreated lignocellulosic material into an alkanol.

17. The process of claim 16 further comprising
neutralizing the pretreated lignocellulosic material before the converting step to a pH in a range of equal to or more than 4.0 to equal to or less than 7.0.

18. The process of claim 17 wherein the neutralizing comprises washing the pretreated lignocellulosic material with water.

19. The process of claim 16 wherein the converting step comprises:
hydrolyzing at least a portion of the pretreated lignocellulosic material to produce a hydrolysis product; and
fermenting at least a portion of the hydrolysis product to produce a fermentation broth comprising the one or more alkanol(s).
